(19) United States
(12) Patent Application Publication
(10) Pub. No.: US 2023/0332140 A1
(43) Pub. Date: Oct. 19, 2023

(54) LAYERED CODING ARCHITECTURES FOR NUCLEIC ACID MEMORY

(71) Applicant: Molecular Assembiles, Inc., San Diego, CA (US)

(72) Inventors: Matthew T. HOLDEN, San Diego, CA (US); J. William EFCAVITCH, San Diego, CA (US)

(21) Appl. No.: 18/042,862

(22) PCT Filed: Aug. 25, 2021
(86) PCT No.: PCT/US21/47508

§ 371 Date: Feb. 24, 2023

Related U.S. Application Data
(60) Provisional application No. 63/069,975, filed on Aug. 25, 2020.

(51) Int. Cl. C12N 15/10

(52) U.S. Cl. CPC C12N 15/1068

(57) ABSTRACT

Described herein are approaches allowing the storing of data at lower densities and increased write speeds. Indexing and recording of data may be separated into separate processes. Rapid DNA extension reactions can then be performed at many distinct locations throughout a solid support, so that the write speed is limited by the ability of the instrumentation to perform spatial addressing operations, rather than chemical synthesis steps.

### CROSS-REFERENCE TO RELATED APPLICATIONS

This application claims the benefit of U.S. Provisional Application No. 63/069,975, the content of which is incorporated by reference in its entirety.

### FIELD OF THE INVENTION

The invention relates to technologies for storing data in sequences of nucleic acids.

### BACKGROUND

DNA has been considered an attractive media for data storage for several decades. Advantages include its minimal physical footprint and energy consumption required for maintenance, the availability of molecular biology techniques to copy existing DNA sequences, and the ability to perform selective isolations from a complex and disordered mixture. There has been a massive increase in the amount of digitally stored data over the past 20 years, as well as substantial advances to DNA sequencing technologies. These developments have spurred a renewed interest in DNA data storage schemes, particularly for large-scale archival purposes. It is now widely held that the speed and cost of the ‘writing’ step is the primary bottleneck limiting further adoption of this technology. These slow writing speeds have arisen largely due to the emphasis on developing DNA synthesis for life science applications, which has led to a mismatch between current synthesis chemistries, coding schemes, and the engineering needed to realize data storage at the exabyte scale.

Storage of even 10 GB (10¹⁰ bytes) of data in chemically unmodified DNA requires 40 billion data encoding nucleotides (nt) (see FIG. 1). Column 1 shows the number of bits in a single data strand, while column 2 gives the coding scheme (binary is base 2). Columns 3-6 then calculate the number of data strands needed to encode 10 gigabytes of data, while columns 7-8 estimate the upper bound on the number of instrument cycles needed to write this data. Note that in the 1-bit cases (bold) the instrument cycle value is one fewer than the (addition cycles*base encoding) because the absence of an operation at a site can be used in lieu of an instrument cycle. Column 9 estimates the amount of time allowed for each instrument cycle to write 10 GB in 24 hours, while column 10 gives the rate at which the instrumentation must perform the spatially selective reactions such as reagent delivery or localized photolysis.

The longest DNA molecules that are chemically synthesized using phosphoramidite chemistry are generally between 100 and 200 nt in length. Accordingly, between 200 and 400 million distinct oligonucleotide sequences are needed to store that exemplary 10 GB data without accounting for the use of index elements or error correction techniques. This poses a problem of scale for current generations of oligonucleotide synthesizers, all of which generate sequences in a stepwise cycle of base-by-base addition to a solid support.

Some techniques make multiple sequences in parallel by performing spatially selective reactions at different sites on the surface. This is typically accomplished by mechanical delivery of individual monomers to the reaction site or through controlled deprotection reactions using light exposure or electrochemical techniques. There are few currently available piezo or inkjet delivery solutions capable of performing 40 billion deliveries with the accuracy needed for data recording within a practical time period or device footprint. Instrumentation which directs the synthesis by controlling deprotection steps need to flood the entirety of the reaction cell with reagents that react only at a subset of the locations on the solid support. This means that the number of instrument cycles is proportional to both the length of the data sequences as well as the number of nucleotide analogs used for the encoding. The effect is that compressing the data through expanded encoding with unnatural nucleotide analogs does not enable significantly faster writing speeds. FIG. 1 provides a more detailed illustration of these tradeoffs. Further, the number of unique sequences required for large scale data storage exceeds the number of reaction sites that can be addressed in parallel on existing oligonucleotide synthesizers.

### SUMMARY

The invention provides methods for enhancing the write speed of DNA-based data storage systems with a layered coding approach. The strategy allows the generation of materials, such as planar wafer surfaces, that are patterned with nucleic acid molecules independently of performing data writing operations on the material. The patterned sequence specifies a physical address within the material and acts as an index for the data written at that site. Each location can be identified by a unique index sequence and may contain one or more indexed initiators upon which data-encoding nucleic acid sequences can be synthesized. The number of indexed initiators per discrete location may be dictated by the error rates of the synthesis (writing) and sequencing (reading) methods used and the associated storage density as discussed below.

Due to the redundancy of multiple indexed data strands at any given location and the encoding schemes described herein, it is not necessary to alter the entirety of the molecules at that location. Accordingly, faster synthesis/writing techniques can be used without the need for 100% incorporation at a given site. When the nucleic acid molecules are cleaved from the material and sequenced, the data writing operations can be associated with the location that the writing operation, such as an enzymatic extension, occurred. The library of nucleic acid molecules acts as a disordered yet compact record of a physical storage media, such as an optical disk drive or holography plate. Aspects of the invention may include techniques for reorganizing and storing the barcoded DNA, concatenation or size selection of DNA oligonucleotides to reduce the number of sequencing reads required to interpret the data, surface preparation techniques, as well as techniques to copy an existing indexed recording medium in a molecular analogy to an imprinting process. While preferred embodiments utilize enzymatic techniques for DNA synthesis, many concepts, coding strategies, and writing approaches can also be applied to oligonucleotides synthesized with phosphoramidite chemistry. The combined effect of these coding improvements and enzymatic processes is such that the write speed is increased by several orders of magnitude at the consequence of a less than 10-fold reduction to storage density.

### BRIEF DESCRIPTION OF THE DRAWINGS

FIG. 1 shows the effect of various data encoding strategies on write speed.

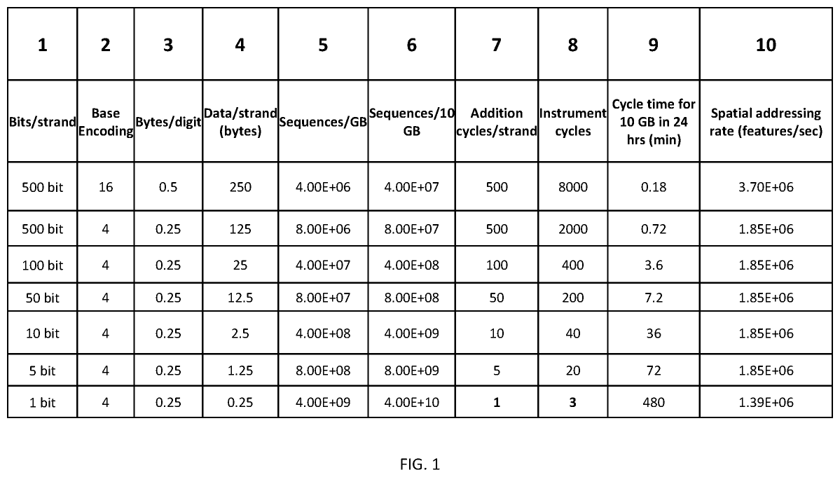

FIG. 2 shows a layered coding strand architecture. The layer boundary may be any predefined nucleotide or combination of nucleotides.

FIG. 3 shows a categorization of various types of data writing steps.

FIG. 4 exemplifies the writing of a layer with an enzymatic class I approach.

FIG. 5 exemplifies the writing of a layer with an enzymatic class II approach.

FIG. 6 shows exemplary mechanical delivery of sub-stoichiometric reagent quantities.

FIG. 7 exemplifies the writing of a layer with an enzymatic class III approach.

FIG. 8 exemplifies the writing of a layer with a class IV approach.

FIG. 9 shows a controlled concatemerization process for archival.

FIG. 10 illustrates recovery of data from sequencing reads by writing class.

FIGS. 11A and 11B show modeling of the depth penalty required for adequate data recovery. FIG. 11A shows the expected error in distinguishing deliberate null reaction events from those arising due to sub-stoichiometric additions during the writing process. Increases to both fractional reactivity or sequencing depth reduce the probability of mis-assigning a bit. FIG. 11B shows the depth penalty required for a 95% confidence in null reaction assignment as a function of the fractional reactivity.

FIG. 12 shows a model of the storage density of a class I system. The chart plots the change in storage density according to the indicated expression. The inset shows the expected distribution of nucleotide additions at an index site from a single extension reaction used in this model. The expected nucleotide addition for this distribution (d), is calculated as weighted average of these addition products (d=1 here, and fractional reactivity f−0.75).

FIGS. 13A and 13B show variations in storage density of a class I system. FIG. 13A models the effect of several addition reaction profiles on the storage density in a class I system. FIG. 13B shows the effect of using an expanded library of unnatural nucleotide analogs storage density. The model assumes 1024 write cycles (and thus 512 addition reactions/index).

FIG. 14 shows exemplary size selection principles.

FIG. 15 shows an effect of size selection on storage density.

FIG. 16 shows surface pattern transfer amplification. First primers are annealed and extended to copy the ‘template wafer’ (bottom surface) sequences. The newly copied strands are then melted off and allowed to diffuse before the surfaces are cooled so that some may anneal to the primers on the ‘blank’ wafer (top). The primers are then extended using the hybridized strand as a template, copying the sequence found on the original surface. The duplexes are then removed so that both surfaces contain indexed ssDNA initiators usable for subsequent writing operations.

FIG. 17 shows a summary of various potential data storage workflows compatible with layered coding approaches.

### DETAILED DESCRIPTION

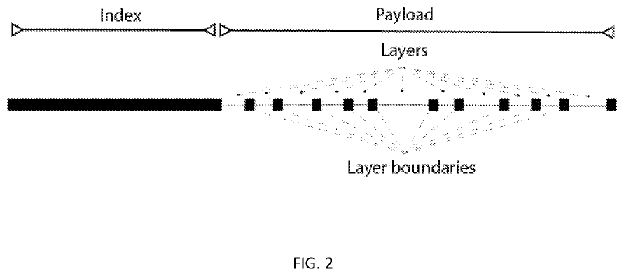

The core of the invention is to enhance the write speed by minimizing dependence on the high-fidelity synthesis steps currently required for all existing ‘storage by synthesis’ techniques. The approach is to use the layered coding architecture shown in FIG. 2. The data strand contains an index region specifying a unique physical address within a recording media. The payload region is generated using an alternating pattern of spatially selective reactions to encode the data and global reactions which introduce boundaries organizing the data into layers. Only the index region and the layer boundaries, shown as the bolded bands in FIG. 2, require high-efficiency synthesis steps. The spatially selective reactions themselves may be sub-stoichiometric, wherein only a partial fraction of the molecules at an index or intended reaction site are modified. This results in a complex library of molecules for each index site. Upon sequencing, the data is recovered by the detection of the presence, absence, or quantity of nucleotides within the data layers denoted by each boundary (see ‘Decoding’). While enzymatic DNA synthesis techniques are preferred for such reactions and discussed in further detail below (see ‘Writing Strategies’), this coding approach may also be used with phosphoramidite chemistry and no description is intended to limit the scope of the invention to solely enzymatic embodiments. A secondary advantage of using enzymatic chemistry is that it allows extensions upon DNA sequences which do not contain protecting groups thereby enabling new workflows for generating and recycling index elements that further reduce the need for in situ chemistry. These strategies are discussed below (see ‘Index generation’).

### Writing Strategies

The data recording steps are performed by using a series of spatially selective reactions upon a suitable substrate or recording media. These may be of any type that can be induced by a write head capable of accessing the location of each unique index site within the media, preferably by physically translating over a larger surface. Suitable write-heads include high-frequency deposition or printing systems, electrode arrays, optical components such as lasers, digital micromirror devices (DMDs), liquid crystal masking systems, or any suitable combination thereof as employed for photolithography, interference lithography or holography. Each spatially selective operation results in the localized addition of one or more nucleotides to a subset of the oligonucleotides immobilized on a two or three-dimensional solid support. Preferred embodiments conduct this addition enzymatically with a template independent polymerase, such as terminal deoxynucleotidyl transferase (TdT), polymerase theta (pol Θ), or a closely related variant to append the nucleotides to the 3′-termini of the surface features. Exemplary template-independent polymerases and template-independent synthesis techniques that may be used with systems and methods of the invention are described, for example, in U.S. Pat. Pub. Nos. 2020/0190491, 2018/0274001, 2018/0305746, and 2019/02755492, the content of each of which is incorporated herein by reference.

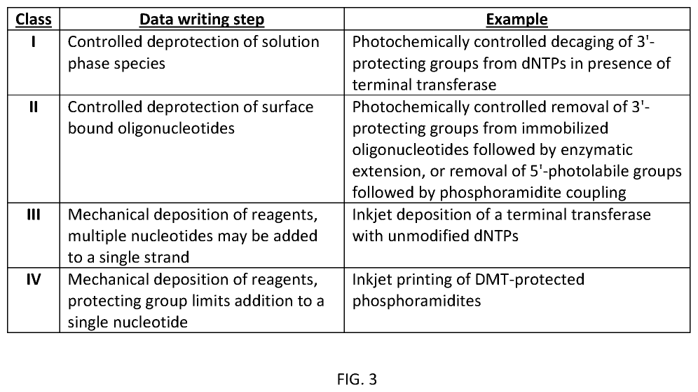

The writing approaches compatible with this coding approach can be divided into four classes depending upon the combination of chemistry, enzymology, and engineering employed. These are summarized in FIG. 3. Classes I and II are defined by instrumentation wherein the spatially selective reaction is the removal of a protecting group which otherwise limits nucleotide addition. In class I embodiments, the protecting group is located on a solution-phase species, such as a modified nucleotide, so that nucleotide addition to cannot occur until its removal. In Class II embodiments, the protecting group is instead covalently linked to the immobilized oligonucleotide, preferably to the 3′-hydroxyl, limiting or preventing extension until its removal. Both Class III and Class IV are defined by instrumentation wherein the spatially selective reaction is the mechanical deposition of the reagents needed for nucleotide addition. In Class III embodiments, a single deposition event may result in the addition of multiple nucleotides to an oligonucleotide within the reagent landing zone. In Class IV embodiments, protecting groups are used so that a single deposition event will not produce extensions greater than a single nucleotide. Note that these classes and examples are for categorization only and some embodiments may utilize combinations of strategies and chemistry from other classes, particularly where employing multiple types of protecting groups can modulate the behavior of enzymatic extension reactions.

In class I embodiments, the write cycle may be conducted by flooding the oligonucleotide-patterned recording media with a mixture of enzyme, buffer, and protected dNTP. The protecting group may be of any type which prevents enzymatic extension and may be removed by any spatially selective techniques known to those skilled in the art such as photolysis, a pH change, or change in oxidative or reductive potential. Nucleotide analogs and protecting groups compatible with the systems and methods described herein are described, for example, in U.S. Pat. Pub. Nos. 2020/0190491, 2018/0274001, 2018/0305746, and 2019/02755492. Spatially selective reactions are then performed to remove the protecting group from the dNTP, so that the decaged molecules become substrates for enzymatic addition to the local oligonucleotide sequences. The occurrence or absence of a spatially selective reaction (a ‘null reaction’ hereafter) encodes for a bit of data depending on the precise coding approach that is used. The enzymatic extension reaction may be stopped by quenching, heat killing, or may instead proceed until the vast majority of substrate molecules have been consumed. The media is then flushed to remove traces of reactive material, completing a single write cycle. These steps are then repeated by utilizing a different dNTP for each subsequent write cycle without recycling the type of dNTP. The number of consecutive write cycles may be limited by the number of dNTPs which can be uniquely identified by a given sequencing technology. For a set of N distinguishable dNTPs, up to N−1 consecutive write cycles may be employed. After N−1 cycles, the remaining dNTP is used in a global addition reaction, where it is added to every molecule in the recording media to denote a boundary of a data layer, thus refreshing the ability to use dNTPs employed in previous write cycles. The order in which the dNTPs are added may be known and fixed (e.g. A, C, G, with T defining the layer boundary) for each layer to aid in decoding/reading. In some instances, it may instead be useful to designate two dNTPs for use as layer boundaries, particularly in embodiments where the length of the layer boundary may not be limited to precisely one nucleotide. This enables reads containing adjacent layer boundary nucleotides to be interpreted correctly.

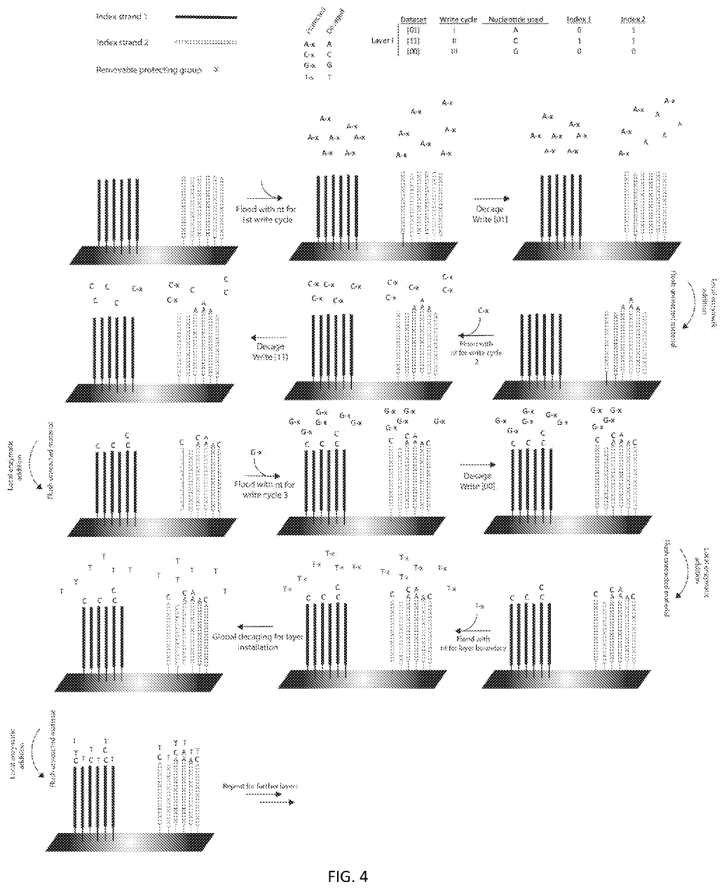

FIG. 4 shows an illustration of writing a complete layer with an enzymatic class I embodiment. Here, the first write cycle is conducted by flooding a reaction cell with a protected ‘A’ nucleotide and the enzyme required to produce extension upon protecting group removal. The spatially selective reaction then removes the protecting group at the second index site, denoting the writing of a 0 bit at the first site (a null reaction), and a 1 bit at the second site [01]. This results in the enzymatic addition of A nucleotides to the local index strands at the second site. The reagents are then flushed from the flow cell, completing the write cycle so that the process may be repeated. The second write cycle is conducted by flooding the reaction cell with protected C nucleotides and enzyme. Deprotection reactions are performed at both index sites to write [11], resulting in the addition of C nucleotides to some strands at each index site, and reagents are again flushed away prior to the next write cycle. The third write cycle is conducted by flooding with protected G nucleotides and enzyme, and no deprotection reactions are performed, thus writing [00]. No addition of G nucleotides occurs as a result. After three write cycles, the recording media is flooded with protected T nucleotides. Deprotection reactions are conducted across the entire media so that at least one T is added to each sequence present, thereby denoting the boundary of the layer.

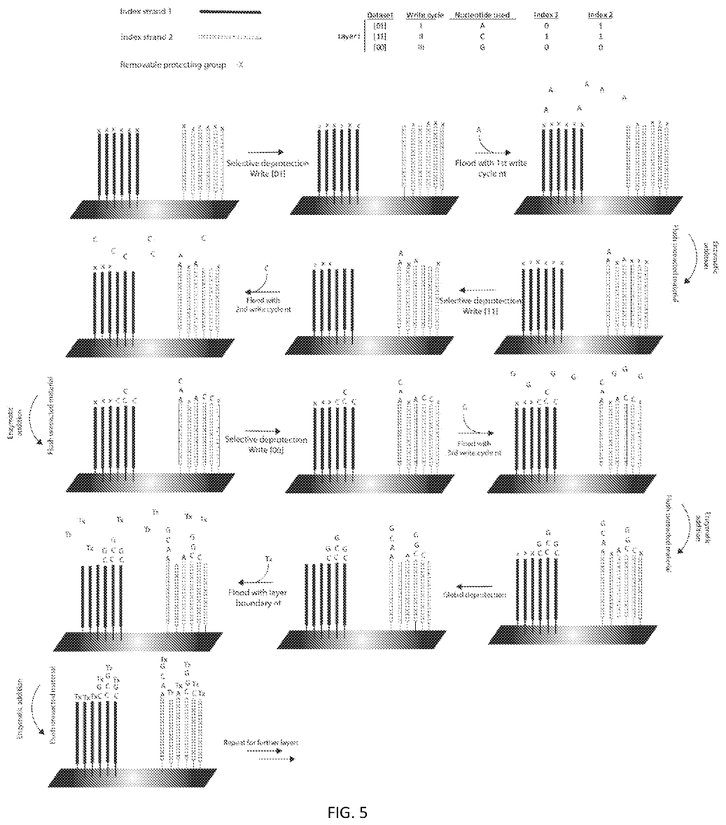

The class II write cycle is conducted upon a recording media where the oligonucleotides contain a protecting group that prevents or significantly limits extension. In preferred embodiments, this protecting group has been installed by performing a global addition reaction to add a protected dNTP to every site in the recording media. The protecting group may be of any type which modulates enzymatic extension on the subsequent cycle and may be removed by any spatially selective techniques known to those skilled in the art such as photolysis, a pH change, or change in oxidative or reductive potential. Localized exposure to the removal conditions render the DNA at an intended site reactive to subsequent extensions. Removal conditions may be selected so that they remove a precise fraction of the protecting groups at a given site. The recording media is then flushed with a mixture of enzyme, buffer, and triphosphate to append a nucleotide or short series of nucleotides to these reactive sites to complete a write cycle. This process may then be repeated by using a different dNTP for each subsequent write cycle without recycling the type of dNTP. When the entirety of the protecting groups at the reaction sites are depleted or there are no remaining unique dNTPs, the reactivity of the surface can be refreshed by another global addition of the protected triphosphate to complete the layer and allow the process to be repeated. FIG. 5 shows an illustration of writing of a layer with an enzymatic class II embodiment and steps of partial photolysis for protecting group removal.

The first write cycle is conducted by performing a partial photolysis reaction only at the second index region, thereby writing a 0 bit at the first index and 1 at the second bit [01]. The reaction chamber is then flooded with A nucleotides and enzyme so that the strands lacking a protecting group undergo extension. The reagents are then flushed away to complete the write cycle. In the second write cycle, both index regions undergo a partial deprotection reaction, writing [11], before the reaction chamber is flooded with C nucleotides and enzyme. This adds nucleotides to the newly deprotected strands at both sites and may also produce extensions on strands which reacted in the first write cycle. The chamber is then flushed prior to the third write cycle. The data written in the third cycle is [00], so no deprotection reactions are conducted. When the chamber is flooded with G nucleotides and enzyme, only the strands which had previously undergone an addition will react. The reagents are again flushed from the chamber, completing the third write cycle. A global deprotection reaction is then conducted to remove all residual protecting groups from the strands before the media is flushed with a T nucleotide that contains a photolabile protecting group and an enzyme to catalyze addition. Enzymatic addition of this T to all the sequences installs the layer boundary and refreshes the photosensitivity of the surface for writing the next layer.

As with class I, the number of nucleotides or nucleotide analogs that can be used within each data layer is limited in part by the ability of the sequencing technology to distinguish the various analogs from one another.

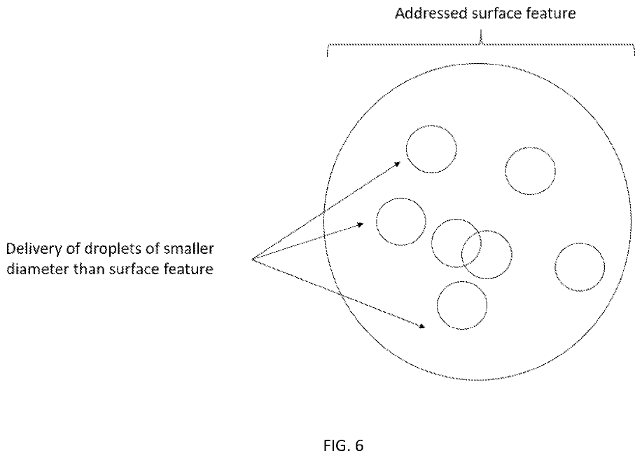

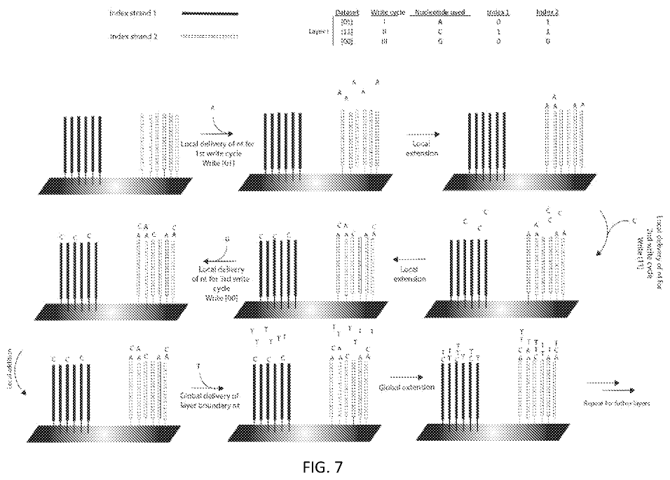

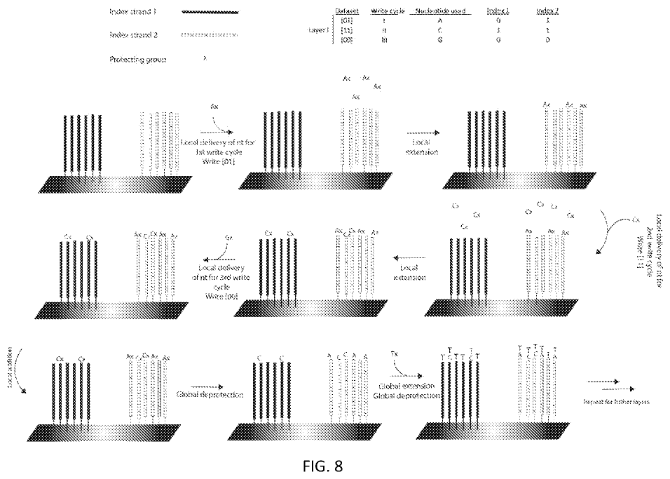

A write cycle for both class III and class IV embodiments require only the selective placement of the necessary reagents at a reaction site in the recording media. Any mixture of reagents capable of inducing a nucleotide addition to immobilized strands may be used. Unlike approaches that repurpose gene synthesis instrumentation for data writing, feature sizes may be larger than the delivery zone of the instrumentation (FIG. 6). This allows either a precise fraction of the surface molecules to react at each cycle or high frequency depositions that do not require precise spatial registration. In class III embodiments, the nucleotide monomers are capable of some degree of polymerization and an oligonucleotide molecule within the delivery zone may undergo more than a single addition event. The monomers in class IV embodiments instead contain a protecting group which prevents multiple extension events from occurring. A class III reaction mix may be comprised of a template independent polymerase, buffer, and dNTPs unmodified at the 3′-hydroxyl. A class IV reaction mix may be comprised of mixtures of a template independent polymerase, buffer, and a dNTP containing a 3′-protecting group, or alternatively a mixture of phosphoramidites and activating agent. After the extension reaction has occurred, the media is flushed, and any intermediate processing steps (such as oxidations or functional group removal) are conducted to complete the write cycle. In some embodiments, it may be possible to proceed directly to the next write cycle without extensive washing. These steps are then repeated using a different dNTP for each subsequent write cycle without recycling. When are either no unique dNTPs or few of the original reactive sites remaining at each index location, a global addition reaction is conducted to complete the layer. In class IV embodiments, it may be necessary to remove residual protecting groups prior to the installation of this layer boundary. FIGS. 7 and 8 show illustrations of writing of a complete layer for a class III and class IV embodiment respectively.

In the first write cycle of the exemplary class III embodiment shown in FIG. 7, a mixture of A nucleotide and enzyme is physically deposited at the location of the second index site. This adds nucleotides of A to at least some molecules at the second index site and not the first, thereby writing [01]. Some strands may undergo the addition of multiple nucleotides. Flushing may be conducted between write cycles in some embodiments but may be omitted in others. In the second write cycle, C nucleotides and enzyme are added to both index sites in the second write cycle to write [11]. In the third write cycle, no depositions of reagents occur to write [00]. The layer boundary is then installed with a global delivery of T nucleotides and enzyme so that strand undergoes at least one addition.

In the exemplary class IV embodiment shown in FIG. 8, the first write cycle is conducted by depositing a protected A nucleotide at only the location of the second index strand, thereby writing a zero at the first index site and a 1 at the second. The protecting group on the nucleotide limits the extension to no more than one nucleotide per molecule. In the second write cycle, protected C nucleotides are delivered to both index regions to write [11]. In the third write cycle, protected G nucleotides are not delivered to either index site to write [00]. A global deprotection reaction is then performed to remove all protecting groups present so that all molecules may undergo the global addition reaction of the protected T nucleotide which denotes the layer boundary. The protecting group on the T nucleotide is then removed to refresh the oligonucleotide reactivity for subsequent layers.

The coding architectures described herein provides several advantages over nearly all existing storage-by-synthesis technologies. First, the approach enables a greater quantity of data to be written with each instrument cycle. In a light-directed array synthesizer utilizing four nucleotides and operating in a synchronous mode (see A. Kahng, I. Mandoui, S. Reda, X. Xu, A. Zelikovsky, “Design Flow Enhancement for DNA Arrays,” Proceedings of the 21st International Conference on Computer Design (ICCD'03), 2003, incorporated herein by reference), each payload nucleotide type will encode the equivalent of 2 binary bits of data and a single addition will occur once at each site in 4 cycles, thus writing at a rate of ½ bit/cycle/site. By contrast, a layered coding approach utilizing one nucleotide type (i.e. T) as a layer boundary and 3 nucleotides (i.e. A, C, and G) within the data layers may write at a rate of ¾ bit/cycle/site. Part of this gain results from capability to use the absence of a reaction, or a ‘null reaction’ to encode information (a 0 bit for example). Second, the approach reduces write time within each synthesis cycle by eliminating the requirement that each addition reaction progress to completion, reducing the dwell time that a write-head such as a laser, micromirror grid, liquid-crystal masking system, or mechanical deposition apparatus, must spend at each location while traversing a recording media. Many protecting groups employed for DNA synthesis exhibit first-order photolysis kinetics and up to seven half-lives may be required to achieve near complete photolysis (Agbavwe, C., Kim, C., Hong, D. et al. Efficiency, error and yield in light-directed maskless synthesis of DNA microarrays. J Nanobiotechnol 9, 57 (2011), incorporated herein by reference). Embodiments which utilize partial (i.e. 20%) photolysis for each reaction may therefore easily realize order of magnitude improvements to write speed. Third, the sub-stoichiometric additions may either be imprecise, wherein the extent of reaction is not crucial to decoding the data and each write cycle encodes information in binary, or precise, where the exact extent of the reaction encodes information at bitrates above binary. Both scenarios are described in further detail below. Fourth, the reduced dependence on high-fidelity synthesis may enable drastic cost reductions in reagents, such as enzymes, triphosphates, or phosphoramidites, particularly at the unprecedented scale required for nucleic acid memory systems. When coupled with the low write-head dwell time, this may in turn enable data storage devices with physical footprints and write speeds closer to that of modern optical drives than existing DNA synthesizers. Sixth, separating the index and payload writing reduces dependence on in situ synthesis techniques, enabling new routes for generating, copying, or recycling indexed recording media, each of which are discussed further below.

### Library Preparation

In some instances, the DNA molecules may remain bound to the support for long term archival. In other embodiments, the DNA may be removed from the support by the aminolysis of an alkaline labile linker, sequence specific enzymatic digestion with uracil specific excision reagent (USER) or a restriction enzyme, or other techniques known to a skilled artisan. The method and technique for the DNA removal from the solid phase is dependent solely on the covalent linkage chemistry and the subsequent processing steps required for archival.

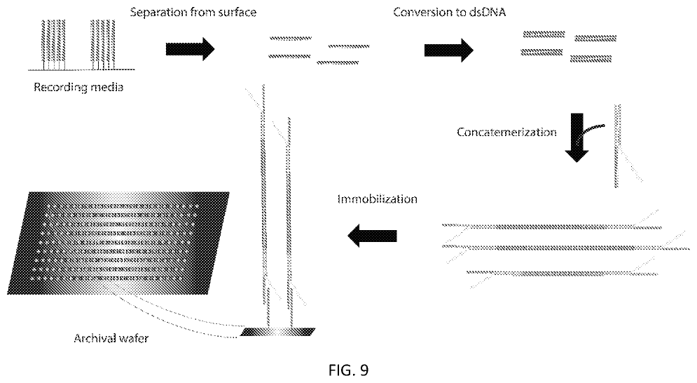

The DNA may be made more amenable to reading by first converting the single stranded data strands to double stranded DNA (dsDNA) and concatamerizing the short duplexes into larger constructs. The conversion to dsDNA can be accomplished with random priming strategies or by installing a common 3′-primer binding site to the data strands after their cleavage from the surface. Preferred embodiments utilized 5′-phosporylated primers so that the short dsDNA products can be used directly in a blunt-end concatamerization reaction. The average length of the concatamers can be tailored by reaction time or varying the ratio of data strands to duplexed adaptors like those used in commercial sequencing library preparation kits, which act as end groups to stop chain growth. The adapters may be either entirely double-stranded to facilitate amplification of the concatemers or partially single-stranded to enable sequence-specific immobilization on an archival surface. The effect is that many short molecules which would otherwise comprise a single sequencing read are assembled into larger constructs, reducing the total reads necessary for sequencing. Appending a barcode to each dataset allows them to be archived together in a smaller physical footprint than the original recording media. Attachment of sequence-specific immobilization handles allow data to be reorganized by new physical addresses upon archival wafer arrays, in turn facilitating selective access to subsets of data within a larger archive. Such archives can be comprised of surfaces patterned with oligonucleotide sequences to selectively capture the dataset barcodes at a defined site. The surface capture sequences may be designed to be orthogonal to one another by any technique known so that only the intended sequences are localized in the intended site. The datasets may then be accessed by any technique that permits selective access to surface features, such as photolysis of the material from the surface when the archival addresses contain a suitable linkage, sequence specific cleavage, toehold-mediated release, or approaches for physical separation that rely on precise mechanical handling. FIG. 9 shows an overview of such an archival workflow

### Decoding

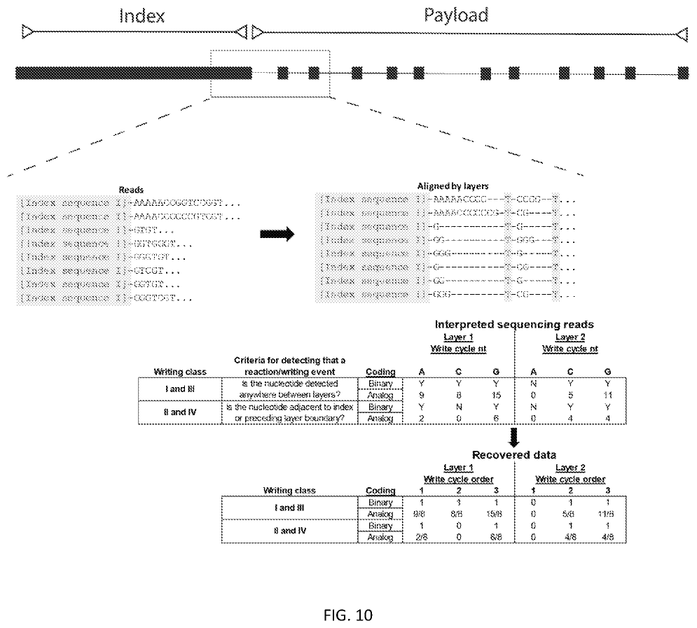

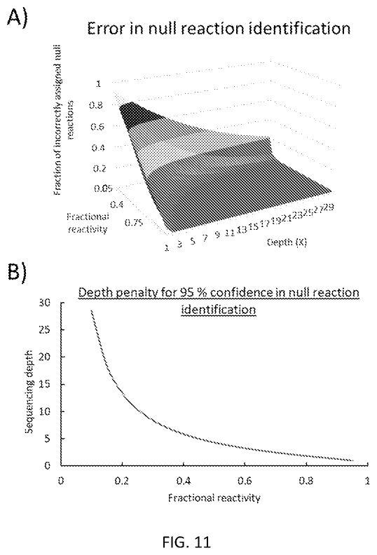

The sequencing of a given dataset may be conducted by any number of current techniques, though long-read, single molecule sequencing approaches such as single-molecule real time sequencing or nanopore technologies are preferred. The only restriction on sequencing modality is that the read length be sufficient to encompass the data payload and index region. The data is recovered by first identifying sections of reads that correspond to index sequences then grouping the payload elements by their affiliated index. The payload sections for each index are then aligned by the nucleotides which denote the layer boundaries. Within each layer, the identity of a specific nucleotide is associated with a specific write cycle. These coordinates of the index, layer, and write cycle uniquely identify the placement of each bit within the full dataset. The bit values themselves are assigned by examination of the nucleotides within each layer to detect whether a spatially selective reaction was conducted. There are distinct ways of making this determination which depend on the writing strategy used. In both class I and class III writing strategies, the occurrence of a writing operation is indicated by the presence of a given nucleotide at any position within the layer. In class II and class IV writing strategies, a writing operation is indicated only by the presence of a nucleotide adjacent to the preceding layer boundary or index. Analog embodiments are also possible, where the frequency of a given nucleotide occurring at a position is assessed relative to the number of reads. FIG. 10 illustrates the read interpretation for each of these scenarios. While the occurrence (i.e. writing a ‘1’) of a data writing operation can be positively determined from individual reads in this manner, the absence of a data writing operation (i.e. a null reaction or writing a 0) is otherwise assumed. The consequence is that a sufficient number of molecules associated with each index must be sampled for this assumption to be accurate. This contrasts with conventional storage-by-synthesis technologies in that there is not a direct relationship between the sequence of an individual molecule and the amount of data it contains. The storage density in nucleotides/bit may be represented in terms of the total number of nucleotides which must be sequenced to recover a given amount of data and is dependent upon the specific writing parameters. The sequencing depth required for data recovery increases if a high fraction of molecules at an index site do not react during the writing steps. If only 10% of the molecules at a site undergo an addition reaction, the sequencing must be of a suitable depth detect such trace reactions with a high probability. This ‘depth-penalty’ is described in more detail in FIG. 11. FIG. 11A depicts the relationship between the fraction of molecules at an index expected to react (the fractional addition), the sequencing depth, and the probability of erroneously assigning a null reaction bit. FIG. 11B depicts relationship between the fractional addition and the sequencing depth needed detect the null reactions with 95% confidence, and thus achieve recovery of >95% the entire data set. Note that this underestimates the recovery because the null reactions comprise on average ˜50% of a full dataset. Some embodiments may operate at very low fractional additions to perform rapid writing reactions, while other embodiments may operate at high fractional additions (nearing or equaling 100% addition) wherein the oversequencing aids in correcting trace errors. Some embodiments may also include parity bits periodically throughout the synthesis to improve error correction capabilities.

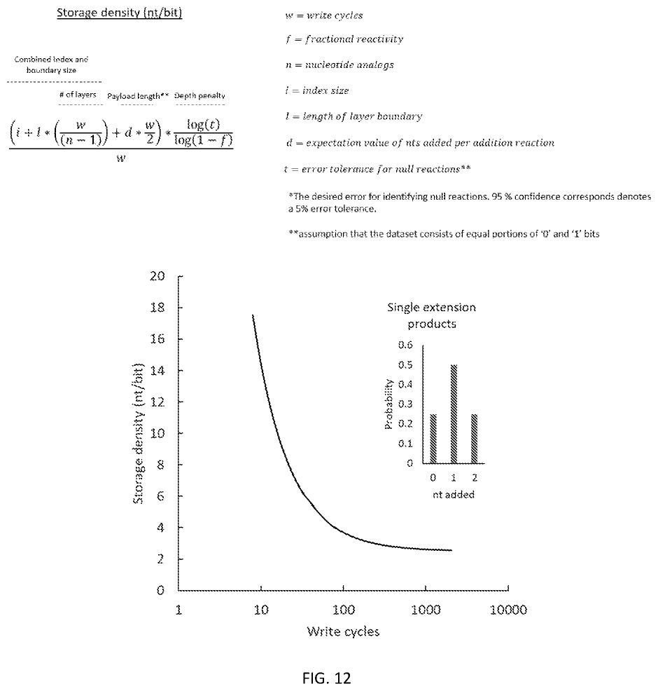

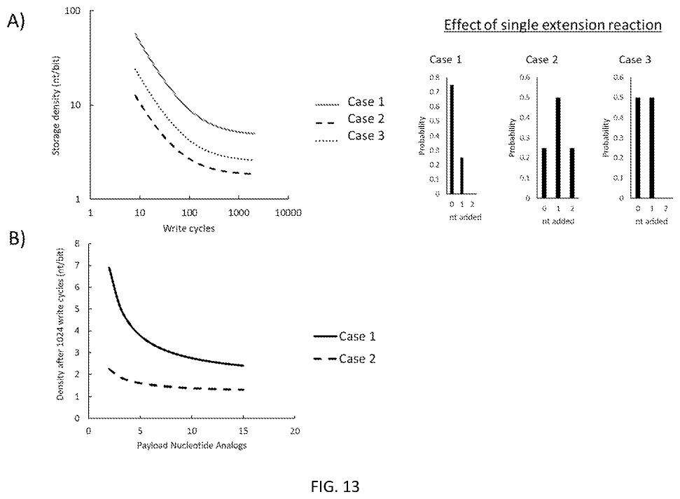

FIG. 12 approximates the storage capacity of an enzymatic class I system. The density is dependent upon the variables shown including the number of analogs, the index length, the number of nucleotides used to form a layer boundary, and the fractional reactivity of the strands undergoing an addition step. Assuming an index 40 nt in length, the use of three nucleotides for the write cycles (dA, dC, dG), with a 4^{th} nucleotide (dT) used to form a layer boundary one nucleotide in length, and a dataset averaging equal parts 0 and 1 bits provides the resultant graph shown in FIG. 12. The inset depicts the distribution of nucleotide additions induced by a single decaging event. The probability of strand receiving not reacting is 25% (fractional reactivity of 75%) which leads to a —2.16x depth penalty (see FIG. 11B). As FIG. 12 illustrates, the depth penalty and index size result in inefficient storage when few write cycles are utilized, but the density rapidly increases with the addition of further layers. FIG. 13A models the impact of various extension reaction profiles on the resultant density with increasing write cycles. FIG. 13B models the impact of utilizing additional nucleotide analogs beyond the naturally occurring dNTPs after 1024 write cycles, illustrating that while some density gains are possible with additional analogs, there are diminishing returns to this approach.

Some embodiments may include size selection strategies to increase the storage density, particularly when lower numbers of write cycles are utilized. These may include capillary electrophoresis, gel purifications, ultrafiltration techniques, selective binding to solid resins, or other chromatographic approaches used by those skilled in the art. The operating principle is that not all strands in a dataset are equally informative. After multiple addition reactions are conducted, there is a distribution of sizes which depends upon the characteristics of the individual addition.

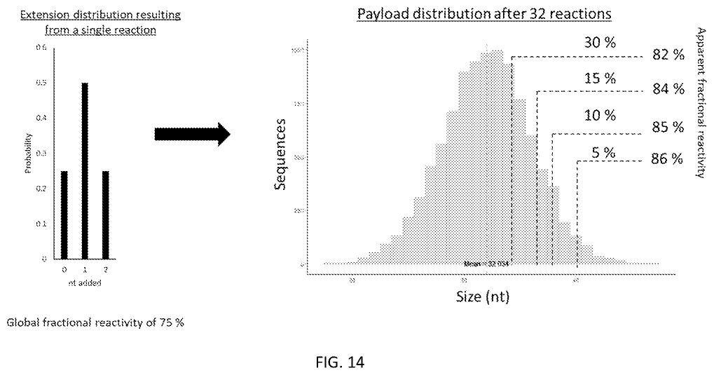

Selection for sequences at various points in this size distribution can alter the apparent fractional addition relative to that of the individual addition as shown in FIG. 14. The left-hand chart shows the extension behavior of a single reaction, while the chart at the right models the distribution of payload sizes after 32 such extension reactions. The fractional reactivity for the entire distribution is 75% but can be increased to higher percentages by sampling as shown.

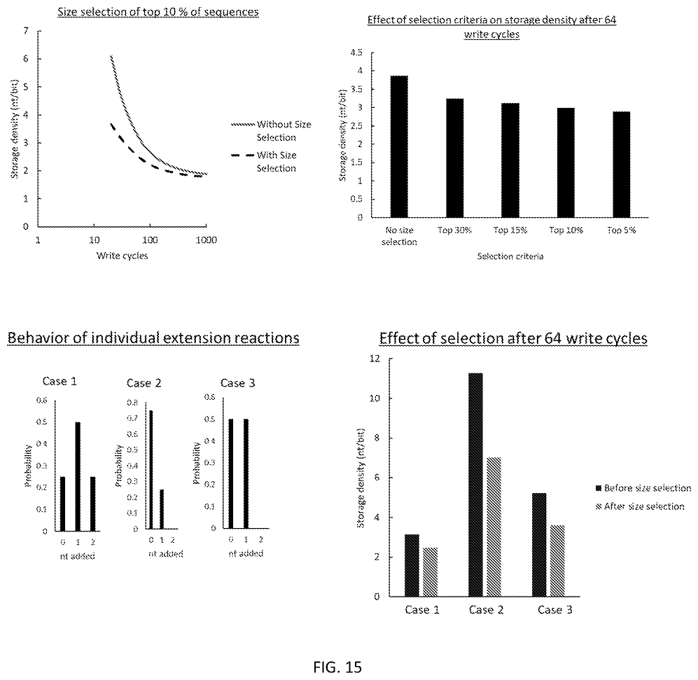

This lowers the depth penalty required for confident identification of the null reaction steps. The resultant gain in storage density may depend on the distribution of the individual extension reactions and the relative size of the data coding regions compared to the index and layer boundaries. FIG. 15 shows the results of in silico size selection model for a class I enzymatic writing system that explores some of these tradeoffs. All density calculations use the same model as shown in FIG. 12. The size selections are modeled by generating in silico payloads as shown in FIG. 14, then correcting for the new average length of the strands after selection and adjusting the apparent fractional reactivity. The upper left-hand figure shows the effect of selection for the largest 10% of data strands with increasing write cycles. Size selection may be uniquely useful for increasing storage density when few write cycles are utilized. The upper-right most chart depicts the effect of the selection criteria on the storage density after 64 write cycles. Both topmost charts assume Case 1 behavior for the single extension reactions. The chart in the lower left illustrates cases of single reaction behaviors, while the chart at the right shows the effect of size selection of the largest 10% of the sequences formed after 64 write cycles. Another assumption is that in 64 write cycles an average of 32 extension reactions occurs.

In some embodiments the order of the write cycles are selected to modulate the properties of the extension reaction. In some embodiments where the fractional addition approaches 100%, it may also be effective to select instead for the smallest oligonucleotides in the distribution.

More complex information can be embedded into each write step of embodiments which utilize photochemically controlled nucleotide addition. Sequencing of a suitable depth can reveal the extent of protecting group removal at each step, so that the intensity of light received at a site can be reconstituted from the sequencing reads and known kinetics of protecting group removal. This allows complex illumination patterns, such holograms and other forms of optical interference patterns, to be encoded and recovered upon sequencing. In class I embodiments, the relative amount of sublayer nucleotide added to each layer encodes the illumination intensity at a given site. In class II embodiments where addition is controlled with a photocleavable group on the surface material, the extent of deprotection at each cycle of addition encodes information about the light intensity utilized.

It is extremely difficult to decipher the encoded data without knowledge of the indexing scheme used during the write steps. Some embodiments may also randomly alter the order of the nucleotide delivery between different layers to further obscure the encoded data. Other embodiments may use additional chemical security features to prevent unintended access or other manipulation of the material. In such embodiments, it is desirable to utilize modified nucleotide analogs which cannot be amplified or copied enzymatically. The material is instead retained as single stranded DNA in an archival system. In contrast to traditional archival systems which emphasize long-term stability, these encrypted data strands may utilize highly labile modifications so that the message will not survive repeated manipulations, attempts at copying, or extended storage.

### Index Generation

A wide variety of surface preparation techniques are appropriate for generation of DNA indexed recording media. The primary requirement is that the material can be engineered to the precision and homogeneity required to achieve a given feature density. In preferred embodiments, feature size is of the same order as the laser spot size (350-700 nm) required to write 10 GB into a surface of comparable size to a compact disk. Accordingly surfaces need to be defect free at this scale to avoid optical defects or disruption to uniform fluid delivery. Suitable substrates include functionalized silicon wafers or silanized glass, polymer sheets, or combinations of surfaces and polymers achieved through spin coating or other deposition techniques known to those skilled in the art. In certain instances, phosphoramidite chemistry may be used to synthesize the index sequences directly from functional groups on the substrate.

In preferred usages, the index DNA is instead generated enzymatically from a universal initiator sequence common to all sites which is covalently linked to the substrate through its 5′-termini. The initiator sequence may contain designed elements to facilitate removal from the surface such as sites for restriction enzyme cleavage, internal deoxyuracil residues, or linkers that are cleaved only under certain conditions such as a specific pH, presence of oxidizing or reducing agents, or exposure to a specific wavelength of light. The selection of covalent linker coupling chemistry is limited only by the orthogonality with the downstream processing conditions so that material is not inadvertently removed from the surface during writing steps. Initiators may be immobilized either so that they are separated by regions of hydrophobicity or so that they uniformly coat the substrate without detectable gaps between features. Any technique which enables the spatially selective extension of the initiators can be used to generate the index strands. Though instrumentation such as mechanical spotting, inkjet-delivery mechanisms, or microelectrode arrays may be used, preferred embodiments utilize lithographic systems such as physical masks, liquid crystal masks, DMDs or any other form of spatial light modulator such as dip pen lithography, so that the extension reactions can be controlled at a suitable resolution. Any coding approach may be used for the indices provided that the sequencing modality can distinguish each index from one another. Some embodiments may utilize a precisely defined nucleotide sequence at each location, while others may utilize a homopolymer-encoded sequence, where the sequence of homopolymer tracts defines the index (i.e. string ‘AAAACCTGGAA’ codes as ‘ACTGA’). Suitable embodiments include those where a triphosphate containing a photocleavable chain terminator is enzymatically added to the 3′-OH of the initiator. Other embodiments utilize caged triphosphates that are prevented from incorporation until a photocleavable group is removed. Suitable molecules may include 3′-ortho-nitrobenzyl protected derivatives, 3′-NPPOC derivatives, nitrobenzyl protected species, or 3′-BODIPY protected species.

Other embodiments may generate indexed surfaces by the random immobilization of complex sequence libraries. Embodiments where index sites are separated from one another and are discontinuous are preferred so that libraries can be hybridized to the surface strands and amplified using primer walking or bridge amplification techniques. Synthesis and hybridization conditions may be selected so that both 1) the probability of 2 identical sequences being present in the library is negligible, and 2) that the probability of two sequences residing at a feature is small. Random template libraries can be synthesized by extending initiator sequences using template independent polymerases and mixtures of dNTPs tailored to reflect the desired base composition. The 3′-end of diverse sequence libraries can be homogenized either through the attachment of a common adaptor sequence or through the enzymatic synthesis of any sequence that allows for primer attachment and/or surface amplification chemistries. Sequencing-by-synthesis approaches can then be used to determine the identity of the indices after immobilization. In other embodiments, composition of the DNA library is known prior to random immobilization so that hybridization-based approaches may be used to decode the location of the strands after attachment. These DNA libraries may also be derived from biological sources of known composition and fragmented, either enzymatically or mechanically, to generate sequences of smaller size and predictable composition. Further approaches may instead utilize the random immobilization and spatial decoding of DNA-coated beads. It may be desirable in some embodiments to employ beads or particles which exhibit distinctive spectral signatures to aid in the decoding. The method of surface preparation does not limit the scope of the invention, in that any approach where the sequence of a polymer or population of polymers can be associated with an address on or within a solid rigid support may be suitable.

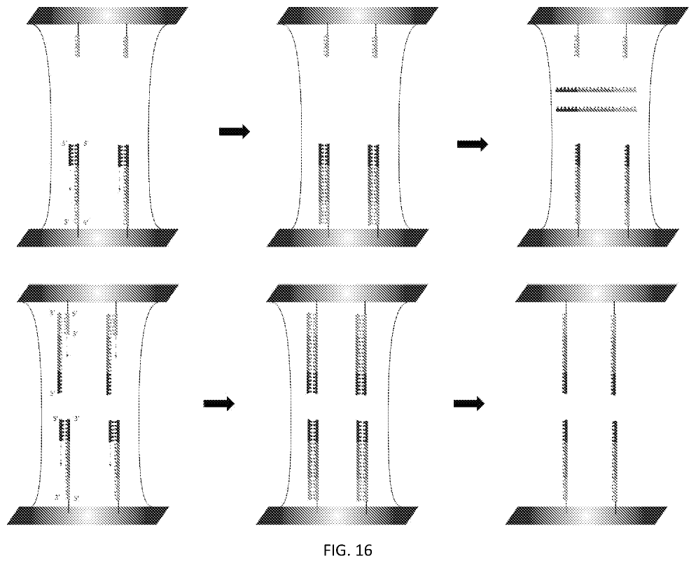

The preparation of such highly indexed materials containing billions of features is the time-consumptive step in the writing process. Accordingly, aspects of the invention may include methods to transfer the pattern of indexes from one material to another. Preferred embodiments for such copying reactions utilize surfaces wherein the features are separated from one another with hydrophobic patches, so that aqueous solutions preferentially form droplets on the oligonucleotide functionalized sites. The features on the master ‘template’ wafer are then registered with those on the ‘blank’ wafer, which are both then pressed into close alignment with one another to form a column of aqueous solution bridging the two features (FIG. 16). A surface amplification reaction is then conducted within each fluid column so that the index sequence from each site is copied from one surface to another. The copying reaction proceeds by annealing a primer the 3′-end of the template wafer, then enzymatically extending the primer to copy the surface bound sequence. The design of the template sequence is such that the 5′-end of the initiator sequence is identical to that on the blank surface. A temperature increase denatures the duplex and allows the newly copied strand to return to solution, where upon cooling, it may either re-anneal to a template site or a location on the blank wafer. The copying step is then repeated. For strands captured on the template surface, another cycle of copies are generated, while strands captured on the blank transfer the sequence information to the new wafer. This cycle is repeated until the necessary surface density is achieved on the template surface. Depending on the design of the initiator sequences used on the template and blank wafers, one or both such surfaces may be used in further imprinting reactions. Aspects of the invention may include performing the copying reaction in humidity-controlled chambers to minimize evaporation and changes to droplet volume during the temperature cycling.

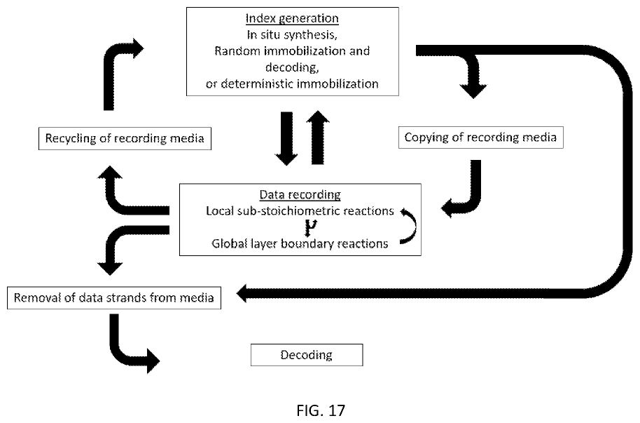

No prior description of the writing strategies is intended to restrict the invention to scenarios where indexing is performed prior to the data writing operations. There are some instances where it instead may be preferable to perform the writing operations before installation of the index elements. In embodiments utilizing enzymatic synthesis techniques, a common initiator sequence may be installed at all locations on the surface so that there is a suitable substrate for a template-independent polymerase. Surfaces may be patterned as previously described, where the initiators form a continuous uninterrupted lawn of oligonucleotides or are localized between regions of hydrophobicity or otherwise passivated region. The data writing operations are performed using any suitable approach as previously described. The index elements may then be installed using any aforementioned approach suitable for appending new sequence information to the 3′-end of the data-encoding strands. In situ enzymatic synthesis may be preferred using similar instrumentation as used for data recording for the subsequent installation of the indices, though it is also conceivable to mechanically deposit and ligate existing indices to the data strands. An advantage of post-write indexing is that surfaces which are relatively easy to prepare can act as a rapid, high-density data recording material that can be indexed and interpreted later at a core archival facility. FIG. 17 summarizes the various workflows enabled by sub-stoichiometric enzymatic reactions.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of storing data in a nucleic acid sequence wherein bits in a dataset are defined by a combination comprising:
an index sequence,
a layer number; and
a presence of a specific nucleotide or nucleotide analog.

2. The method of claim 1 wherein the combination specifying bits in the dataset further comprises an absence of a specific nucleotide or nucleotide analog.

3. The method of claim 1 wherein each of a plurality of nucleic acids at a discrete physical location comprise the same index sequence.

4. The method of claim 3 wherein the recording media comprises a plurality of discrete locations and the plurality of nucleic acids at each of the plurality of discrete locations comprise a unique index sequence.

5. The method of claim 1 wherein the layer number in a DNA sequence is specified by a number of layer boundaries between a region of interest and the index region.

6. The method of claim 5 wherein at least one specific nucleotide is used to indicate a layer boundary in a sequence.

7. The method of claim 2 wherein the presence or absence of each nucleotide or nucleotide analog corresponds to a write cycle.

8. The method of claim 7 wherein each write cycle in each layer corresponds to a distinct nucleotide or nucleotide analog.

9. A system for storing data in nucleic acid sequences, the system comprising:
media comprising a solid support, wherein the solid support comprises a plurality of regions of covalently linked DNA strands, and wherein the covalently linked DNA strands in each of the plurality of regions comprises a unique index sequence.

10. The system of claim 9 further comprising a spatially addressable head operable to deliver one or more of nucleic acid synthesis reagents or decaging stimulus to each of the plurality of regions individually.

11. The system of claim 9 wherein the plurality of regions is in excess of 100 million

12. The system of claim 9 wherein the plurality of regions is in excess of 1 billion

13. The system of claim 9 wherein the plurality of regions is in excess of 10 billion

14. The system of claim 9 wherein the plurality of regions is in excess of 100 billion

15. The system of claim 9 wherein the unique index sequence is generated using in situ synthesis

16. The system of claim 15 wherein the in situ synthesis is performed with a template-independent polymerase from an initiator sequence.

17. The system of claim 9 wherein the unique index sequence is immobilized through mechanical deposition of pre-synthesized sequences onto a surface

18. The system of claim 9 wherein the unique index sequence is randomly deposited from a complex library and amplified to higher densities

19. The system of claim 18 wherein the complex library is synthesized using a template independent polymerase and a mixture of trisphosphates

20. The system of claim 18 wherein the complex library comprises sequence fragments that are isolated from biological sources

21. A method for copying DNA recording media, the method comprising:
preparing a DNA-indexed template wafer comprising DNA-patterned sites separated by hydrophobic regions wherein DNA strands in the DNA-patterned sites comprise an index element flanked by a different primer binding region on both its 5′ and 3′ sides;
preparing a blank wafer comprising DNA-patterned sites separated by hydrophobic regions wherein DNA strands in the DNA-patterned sites contain the same 5′-primer binding site as the DNA-indexed template wafer;
forming droplets at each DNA-patterned site on the DNA-indexed template wafer;
aligning the DNA-patterned sites of the DNA-indexed template wafer with the DNA-patterned sites of the blank wafer such that the droplets form water columns bridging the DNA-patterned sites of the DNA-indexed template wafer and blank wafer;
annealing a primer to the 3′-primer binding site on the DNA-indexed template wafer;
performing template-dependent extension of the primer to copy the DNA strands in the DNA-patterned sites of the DNA-indexed template wafer;
denaturing the copied sequence;
re-annealing at least a portion of the copied sequence to the DNA strands in the DNA-patterned sites of the blank wafer;
performing template-dependent extension on the re-annealed portions of the copied sequence;
repeating the process of denaturation, re-annealing, and template-dependent enzymatic extension steps until a desired oligonucleotide density is achieved on the blank wafer.

22. A method for recording data to a DNA-patterned recording media using spatially selective chemical reactions wherein the occurrence or non-occurrence of a spatially selective chemical reaction is used to encode data as a binary bit ‘1’ or ‘0’

23. The method of claim 22 wherein an extent of the spatially selective chemical reaction encodes data using bit values above base-2 encoding

24. The method of claim 22 wherein the spatially selective chemical reactions are local electrochemical reactions conducted with a microelectrode array

25. The method of claim 22 wherein the spatially selective chemical reactions are mechanical deliveries of a buffered reaction mixture comprising a template independent polymerase and a deoxyribonucleotide triphosphate

26. The method of claim 22 wherein the spatially selective chemical reactions are localized photolysis

27. The method of claim 26 wherein localization of the photolysis reactions is controlled with a spatial light modulator

28. The method of claim 27 wherein the photolysis is conducted with electromagnetic radiation of a wavelength between 300-410 nm

29. The method of claim 27 wherein the photolysis reaction is conducted with electromagnetic radiation of a wavelength between 450 and 700 nm

30. The method of claim 27, wherein the spatially modulated light is generated from the interference pattern of at least two coherent light sources

31. The method of claim 22, wherein the occurrence of a spatially selective chemical reaction facilitates the template-independent enzymatic extension of at least a fraction of oligonucleotides comprising a reaction site with a dNTP

32. The method of claim 31, wherein the spatially selective chemical reaction removes a protecting group from the terminal nucleotide of a surface bound initiator which otherwise prevents extension of that initiator with a template-independent polymerase.

33. The method of claim 31, wherein the spatially selective chemical reaction removes a protecting group from a dNTP which otherwise prevents its polymerization with a template-independent polymerase.

34. The method of claim 31, wherein a plurality of write steps are conducted by utilizing a defined series of dNTPs so that each distinguishable dNTP corresponds to a cycle of data writing.

35. The method of claim 34, wherein the defined series of dNTPs are reused after addition of a distinct intervening region of sequence, the distinct intervening region of sequence serving as a layer boundary and separating layers of encoded data.

36. The method of claim 34, wherein the plurality of write steps are conducted on a DNA-patterned recording media prior to appending unique indices to each location

37. The composition of claim 36, wherein the recording media comprises a solid support patterned with at least one common initiator sequence at every desired synthesis location

38. The method of claim 37, wherein the unique indices are enzymatically synthesized in situ using data encoding strands as initiators after the data has been recorded

39. A method for recovering DNA-encoded data, the method comprising:
sequencing data-encoding DNA strands;
grouping sequencing reads by index sequences;
aligning the grouped sequence reads by layers;
translating an occurrence, extent, or absence of a writing reaction at each write cycle, in each layer, for each indexed group into a bit of data;
combining bits of data from each write cycle, in each layer, for each indexed group to create a dataset.

40. The method of claim 39 further comprising separating the data-encoding DNA strands from a recording media before sequencing.

41. The method of claim 40 further comprising selecting depth for the sequencing step to compensate for partial addition reactions during data writing operations.

42. The method of claim 40 further comprising determining the layers by identifying layer boundary nucleotides.

43. The method of claim 40 further comprising altering sequencing depth by selecting for specific size ranges of the data-encoding DNA strands.
